(19) United States
(12) Patent Application Publication
(10) Pub. No.: US 2004/0247705 A1
(43) Pub. Date: Dec. 9, 2004

(54) Transdermal compositions and methods of treatment to alleviate or prevent migrainous headaches and their associated symptoms

(76) Inventor: Stephen C. Roberts, Minnetonka, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/457,028

(22) Filed: Jun. 6, 2003

(51) Int. Cl. A61K 35/78; A61K 31/522

(52) U.S. Cl. 424/729; 424/756; 514/473; 514/263.31

(57) ABSTRACT

Methods of treating migrainous headaches and their associated symptoms are provided by administering a composition comprising parthenolide or feverfew transdermally to a patient in need thereof. Treatments are surprisingly effective with low total administered amounts of parthenolide or feverfew. Compositions and transdermal administration patches are also described.

### FIELD OF THE INVENTION

The present invention relates to treatment of migrainous headaches and their associated symptoms. The present invention also relates to transdermal treatment regimens and transdermal compositions comprising parthenolide.

### BACKGROUND OF THE INVENTION

The American Migraine Study reports that 32 million Americans, 11.8% of the population, suffer with at least one migraine headache annually, and that 23 million people in the US suffer with “severe migraine”(Stewart WF JAMA 1992; 267:64-9, Lipton, R B Headache. 2001; 41:638-645). Women account for two-thirds to three-fourths of all migraine patients, as gender specific prevalence in the US is 17.6% for females and 6% for males using the IHS (International Headache Society) criteria for migraine diagnosis (Lipton R B, Neurology 1993; 43(Suppl 3):6-10). Twenty-five percent of women with migraine experience four or more severe attacks per month, 35% experience one to three severe attacks per month, and 40% experience one or less than one severe attack per month. Similar frequency patterns of migraine were observed in men (Stewart WF, JAMA 1992; 267:64-9). The overall average number of migraine attacks per year for men is 34 and 37.4 for women. In addition, “chronic recurrent headache” affects approximately 45 million Americans, the underlying pathophysiology for this condition is not yet fully understood (Meuller, L Journal of the American Osteopathic Association, 2000 September;100(9 Suppl):S14-21). Many researchers believe that these clinically significant headaches are primarily migraine-like (migrainous).

Headaches are one of the most common complaints heard by primary care physicians, accounting for over 10 million office visits each year in the United States. Three studies suggest that migraine prevalence may be increasing in the US (MMWR: Morbidity and Mortality Weekly Report 1991; 40:331-8; Stang P E, Neurology 1992; 42:1657-62; Pryse-Phillips W, Can J Neurol Sci 1992; 19:333-9). In fact, it maybe that only the number correctly diagnosed is increasing. A very recent study of 2,524 subjects with “sinus headache” found that a full 90% were actually suffering from migraines (Saper, J. Presentation to American Headache Society, June 2002). The true incidence of migraine cannot be precisely known, but is very high.

Migraine is a severe and disabling condition. More than 85% of women and more than 82% of men with severe headache had some headache related disability (Stewart WF, JAMA 1992; 267:64-9). Approximately 33% were severely disabled or needed bed rest during an attack. Many studies have examined various aspects of headache related disability. Migraine is not just an episodic disease; it is also a chronic disease with episodic exacerbations. Many migraineurs live in fear knowing that an attack will disrupt their ability to work, to take care of their families, and to meet social obligations. Thus, there is some disability between attacks as well as during attacks. Quality of life measurements have shown that migraineurs, compared to those with other chronic illnesses, have lower scores in physical functioning and role functioning, and also experience more body pain (Dahlof, C Cephalalgia 1993; 13:233-7).

Migraine can induce a host of serious physical conditions: strokes, aneurysms, permanent visual loss, severe dental problems, coma and even death. According to the New England Journal of Medicine, “migraine can sometimes lead to ischemic stroke and stroke can sometimes be aggravated by or associated with the development of migraine.” Twenty-seven percent of all strokes suffered by persons under the age of 45 are caused by migraine. Stroke is the third leading cause of death in this country. According to the Mayo Clinic, twenty-five percent of all incidents of cerebral infarction were associated with migraines. Migraine and epileptic seizure disorders are also related. The most intimate relationship between the two being migraine-triggered epilepsy. Migraine affects up to 15% of the epileptic population. In basic terms, migraine and epilepsy are both disorders characterized by short-term, fleeting alterations of neurologic function, usually with normal neurological examinations between attacks.

Finally, the economic burden of migraine indicates its severity, costing the US economy over $14 billion per year.

Economic Cost of Migraine in the US (Hu, X Archives of Internal
Medicine 1999 159: 813-8)

| | US$ million | | |
|---|---|---|---|
| Cost Element | Men | Women | Total |
| Medical | 193 | 1,033 | 1,226 |
| Missed workdays | 1,240 | 6,662 | 7,902 |
| Lost productivity | 1,420 | 4,026 | 5,446 |
| Total | | | 14,574 |

Almost all of those affected by migraine use some medicine for pain, and almost all users of prescription pain relievers are also heavy users of over-the-counter (“OTC” or non-prescription) products. U.S. consumers spend over $6 billion annually on headache pain relievers (OTC and prescription products combined). OTC products used by migraineurs, such as aspirin, acetaminophen, ibuprofen and other common analgesics, exhibit side effects associated with both chronic use and short-term overuse, which may include liver damage, kidney damage, ulcers and stomach upset. These side effects are frequently severe. Each year, use of non-steroidal anti-inflammatory drugs (NSAIDs) such as aspirin and ibuprofen, account for an estimated 7600 deaths and 76000 hospitalizations in the United States (Fries J F. Assessing and understanding patient risk. Scand J Rheumatol Suppl. 1992;92:21-4.). Frequent use of OTC analgesics has been recognized as a substantial contributor to the development of daily headaches (“chronic daily headache”). Rebound headaches are also an issue, especially with products incorporating caffeine or aspirin.

The prescription migraine market in the US for triptans alone (a class of medication frequently used to treat migraines; examples include Imitrex, Zomig, Maxalt, etc.) was approximately $3.2 billion in 2001 (55 million prescriptions). Side effects commonly observed in this class of drugs include chest pain, shortness of breath, palpitations, paresthesias (sensation of burning, warmth, heat, numbness, tightness or tingling), asthenia (unusual tiredness or muscle weakness), dizziness, dry mouth, fatigue, hot flashes, nausea, vomiting, and sleepiness. Less common side effects include joint pain, CNS effects (agitation, anxiety, confusion, depression, irritability), eye problems (blurred vision, dry eyes, irritated eyes), chills, constipation, diarrhea, heartburn, dysphagia (trouble swallowing), euphoria, flatulence, heat sensitivity, hypertension, increased sweating, increased thirst, insomnia, muscle stiffness, muscle pain or spasms, polyuria (increased urination), pruritis (generalized itching of the skin), tinnitus (ringing in ears), tremor, vertigo, and warm or cold sensations. The fact that 55 million prescriptions are written each year in the United States for a class of drugs which cause up to 43% of users to experience adverse effects with any given dose (Geraud, G. Headache 2002 April; 42 Suppl 2:93-9) indicates the desperation of migraine patients and the need for better treatments. Total migraine prescription sales probably exceeded $4 billion and the market is growing at 4% annually (Brown, K The Scientist, 11[11]:1, May 26, 1997).

Less than half of those with migraine (48%) have been appropriately diagnosed, and only 41% of those with known migraine use prescription drugs, 57% relying exclusively on OTC medications for treatment of their migraines (Lipton, R Headache Vol 41, pp638-645, August 2001). The above study also revealed that more than half of all Americans who suffer from migraine headaches are prepared to put up with the pain rather than seek treatment from a physician, and that 26% had stopped seeing their doctor about the condition because they felt the doctor could not help. All of this suggests a heavy reliance on, even a preference for, OTC medication for the treatment of migraine. While there are no hard numbers for OTC migraine medicine sales, total OTC analgesic sales in the United States were $3.49 billion, up +2.6% vs. year prior, for the 52-weeks ending Jul. 15, 2000. OTC headache specific remedy sales were $2.65 billion, up 1.8%, on unit volume of 560 million. The fact that 57% of consumers rely entirely on OTC medications to treat a very painful, recurrent and debilitating disease demonstrates the extent of consumer preference for OTC medicines whenever available. Present OTC medications are limited in both number and effectiveness.

U.S. Pat. No. 6,103,218 to Brucker, et al., issued Aug. 15, 2000 discloses a composition and delivery system for administration of dried leaf particles of feverfew in the form of aqueous nasal spray composition. This mode of administration is stated to provide therapeutic moisturization of nasal mucous membranes, relief of migraine headaches and antispasmodic effect, such as to relieve menstrual cramping or aid digestion.

U.S. Pat. No. 6,312,736 Kelly, et al., issued Nov. 6, 2001 discloses an herbal composition for relief of pain and other symptoms associated with migraines and other types of headaches comprising: from about 46% to about 64% by weight of white willow bark extract; from about 8% to about 18% by weight of Kava Kava root extract; and from about 25% to about 41% by weight of at least one of feverfew extract and ginger root extract. The herbal composition disclosed therein is stated to be applied transdermally for rapid relief of pain.

U.S. Pat. No. 4,758,433 Johnson, et al., issued Jul. 19, 1988 discloses a preparation for pharmaceutical use, especially in the treatment of migraine, arthritis and bronchial complaints that contains a sesquiterpene lactone and is recovered from the plant Tanacetum parthenium by extraction using a pharmaceutically acceptable oil and is also delivered via said pharmaceutically acceptable oil, the oil being selected from the group consisting of saturated and un-saturated long chain hydrocarbons and fatty acids, vegetable and animal oils and polyoxyethylated derivatives thereof and reconstituted glycerides and esters thereof, wherein said hydrocarbon chain comprises between 10 and 25 carbon atoms, especially coconut oil, soybean oil or fish oil.

In addition to prescription and OTC medications, those with migraine headaches have attempted to treat this malady using herbal medicine or homeopathic treatments. Herbal medicine is defined as the use of herbs for their therapeutic or medicinal value. In the United States, herbal products are generally marketed as dietary supplements. An herb manufacturer or distributor can make no specific claims to diagnose, treat, cure or prevent any disease without having first obtained FDA approval. Nonetheless, information of the use of herbs for treatment of various maladies is widely available, including authoritative sources such as the Physicians Desk Reference (PDR) for Herbal Medicines, published by Thompson Medical Economics. Homeopathy is a separate theory of medical treatment, based on principles formulated by Samuel Hahnemann in the late 1700's. The practice of homeopathy is based on the belief that disease symptoms can be cured by infinitesimally small doses of substances which, in larger amounts, produce similar symptoms in healthy people. The Federal Food, Drug, and Cosmetic Act (the Act) recognizes as official the drugs and standards in the Homeopathic Pharmacopoeia of the United States and its supplements (Sections 201 (g)(1) and 501 (b), respectively).

Both feverfew and ginger have a long history of use as homeopathic and herbal remedies. Ginger has not historically been used for headache, but has been used with some success for relief of nausea. The administration of 1,000 to 2,000 mg of ginger orally by tablet has been found to effectively reduce nausea in the case of motion sickness (Lien, H C Am J Physiol Gastrointest Liver Physiol 2003 March; 284(3):G481-9). Ginger was investigated recently for its effect on nausea in morning sickness (Keating, A Altern Ther Health Med 2002 September-October; 8(5):89-91). In this study women took 1,000 mg of ginger in divided doses, the researchers concluding that ginger “may be helpful” in treatment of morning sickness. According to the Physician's Desk Reference for Herbal Medicines, the daily recommended dose of ginger for a variety of gastrointestinal symptoms (primarily various forms of nausea) ranges from 500 mg to 4000 mg. (PDR for Herbal Medicines, Thompson Medical Economics, Second Edition, Ginger, 339-342, 2000.) One published report suggests that ginger may be beneficial in the treatment of headache as it “has been reported in Ayurvedic and Tibb systems of medicine to be useful in neurological disorders.” (Mustafa, T J Ethnopharmacol 1990 July;29(3):267-73). Despite this, there have been no known clinical investigations of low dose ginger, sublingual ginger or ginger in the treatment of migraines or headache in general.

Feverfew is another herb that is widely available and has been investigated in modern times. Historically, feverfew is known to have been used in the treatment of fevers, from whence it derives its name, and also in rheumatic conditions. Feverfew is used in homeopathic remedies, but homeopathy recognizes no role for feverfew in the treatment of headaches. An authoritative homeopathic text is “A Dictionary Of Practical Materia Medica” by John Henry Clarke, M.D., recognized as such by the United States Food and Drug Administration, (see the “Compliance Policy Guide: Conditions Under Which Homeopathic Drugs may be Marketed”http://www.fda.gov/ora/compliance ref/cpg/cpgdrg/cpg400-400.html). The Clarke text gives no indication for the use of feverfew in the treatment of headaches. This same text defines the appropriate preparation of feverfew as “a tincture of fresh leaves.” A tincture is a concentrated herbal extract prepared by soaking an herb in alcohol for an extended period of time. The result is an alcoholic extract referred to by homeopathic practitioners as the “mother tincture.” This “mother tincture” is then subject to numerous serial dilutions with the resulting homeopathic drug being extremely dilute. Classic homeopathic remedies do not rely on any effect from the substance first contained in the starting material (“mother tincture” in this case) and often statistically contain virtually no actual molecules of the original substance (feverfew). Instead, these remedies rely on the “imprint” or “energy” of the original substance to exert an effect. Consistent with the precepts of homeopathy, the remedy thus prepared is felt to become increasingly potent, indeed stronger and more effective, as it becomes more and more dilute. Some of these homeopathic remedies may have been administered transdermally, or may yet be administered transdermally by those presently adherent to the practice. Such purported remedies would certainly contain less than 0.01 mg/ml of parthenolide and would not be employed for headache. It would be surprising to a practitioner in the art of homeopathy not only that feverfew is effective in the treatment of headache, but that the benefit not manifested at the extremely dilute concentrations employed by homeopathy is manifest by the present composition. There is little to no clinical support for any of the multitude of homeopathic remedies. Thus it is not surprising but still noteworthy that no clinical evidence exists for the effective use of feverfew in homeopathic medicine as a treatment for headache, especially as homeopathy does not recognize nor endorse this use.

Herbal medicine, as a field distinct from classical homeopathy, has in fact recognized the potential value of feverfew in the prophylactic (preventative) treatment of migraine. Fresh feverfew leaves have sometimes been chewed by subjects wishing to rid themselves of migraine. However, a common adverse effect reported by those who have used this technique is the generation sores in the mouth and sensitization of oral tissues. Additionally, many patients find this mode of administration to be crude and unpleasant. Dosages of feverfew administered historically and presently employed far exceed those of the present invention. In addition, the role of feverfew leaves in the treatment of migraine has always been restricted to prophylaxis (prevention), never having been applied to the acute treatment of headaches.

In addition to raw leaves, feverfew tablets or capsules have been and are employed by practitioners of herbal medicine. These are widely available in any “health food store” for purchase by the general public. The PDR for Herbal Medicines lists migraine, arthritis, rheumatic diseases and allergies as the indications for feverfew usage (PDR for Herbal Medicines, Thompson Medical Economics, Second Edition, Feverfew, 306-309, 2000.) Several studies published in leading medical journals, including “Lancet” (Murphy, J J Lancet Jul. 23, 1988;2(8604): 189-92), and “The British Medical Journal” (Johnson, E S British Medical Journal Aug. 31, 1985;291(6495):569-73), have suggested a potential role for feverfew in reducing the incidence and/or severity of migraines. The Murphy study administered one capsule of feverfew leaves to be swallowed by the patient, wherein each capsule contained about 2.19 micromoles of parthenolide (about 0.5 mg). The Johnson study administered two capsules of freeze dried feverfew powder every morning. The daily dose was therefore 50 mg feverfew. The parthenolide content of this feverfew powder was not reported. Patients reported a reduction in the number and/or severity of migraine attacks, with no side effects reported by either study. Most recently however, several systemic reviews of feverfew use in the prevention of migraine have been published (Vogler B K, “Feverfew as a preventive treatment for migraine: a systematic review.” Cephalalgia 1998 December;18(10):704-8) and (Pittler M H, “Feverfew for preventing migraine.” Cochrane Database Syst Rev 2000;(3):CD002286) both of which reviews concluded that the efficacy of feverfew for the prevention of migraine “has not been established beyond reasonable doubt.” Pittler also noted that “the trial with the highest methodological quality, which was also among the largest, found no significant difference between feverfew and placebo.” Most clinicians in the United States do not consider feverfew an effective prophylactic treatment for migraine and as such do not endorse its use. Feverfew has not been used for the acute relief of migraine attacks. Each of the studies investigated only its prophylactic use. The use of feverfew acutely, for relief of headaches once they have begun, has not been studied and there is no scientific literature that directly suggests that it might be effective. In addition, the prophylactic effect is not said to be noticeable for some number of weeks (2-12) after having first initiated use of feverfew, regardless of the form of feverfew employed (tablets, leaves, etc.). Recommended dosages of feverfew tablets or capsules are 200 to 250 mg one to three times daily, there being no suggestion that alternate routes of administration or lesser doses might prove beneficial. Quite to the contrary, manufacturers generally emphasize the benefits of larger doses of feverfew. Again, neither the PDR For Herbal Medicines nor any other available herbal literature suggests the use of feverfew transdermally or as treatment of acute migraine attacks, it being believed that feverfew is wholly ineffective by such application for this purpose, or by any application for acute treatment.

A product currently available on the market is sold under the name MigraSpray®, which is stated to be a patented over the counter homeopathic drug intended to be a comprehensive approach for the treatment and prevention of migraine headaches. MigraSpray contains the active ingredients feverfew, polyporus, goldenseal and dandelion. MigraSpray is sprayed under the tongue (sublingual administration), which promotes enhanced bioavailability and rapid absorption by directly entering the bloodstream through the mucous membrane avoiding degradation from exposure to the gastrointestinal tract and liver. Product literature claims that this sublingual delivery system allows MigraSpray to provide rapid relief from migraine headache pain and other associated symptoms in an average of less than 7 minutes. However, this product, like other products sold as “homeopathic” treatments, delivers an extremely low dosage of feverfew. The amount of parthenolide reported to be present in this composition is 0.0112 mg/dose.

Additionally, it is noteworthy that the content of parthenolide in feverfew may vary to a great extent depending on the particular variety of Tanacetum parthenium plant grown, and also the manner of processing the feverfew herb. Parthenolide has been found to be unstable and sensitive to processing. Thus, the collection and processing steps carried out incorporating feverfew into a product may reduce or destroy the parthenolide content of the feverfew. Without a standardization of the parthenolide content of feverfew used in the process and careful control of the manufacturing process, great inconsistency is observed in parthenolide content from batch to batch of product.

U.S. Pat. No. 5,503,843 to Santus discloses a transdermal patch for the delivery of a specific Compound to the skin of a patient. The patch comprises a backing layer, a drug depot comprising the compound and a permeation enhancer composition.

U.S. Pat. No. 5,837,289 to Grasela, et al. discloses a composition and procedures for its formation and administration to provide a convenient, efficacious and simple transdermal administration of medications from a topically applied cream. The composition incorporates at least two separate penetration enhancers which function synergistically to provide for rapid but controllable transport of the medication from the cream into the skin. The use of a plurality of penetration enhancers, at least one of which facilitates the separation of medication from the cream and at least a second of which alters the structure of the outer layers of skin, particularly the stratum corneum, enhances migration of the drug through the stratum corneum.

U.S. Pat. No. 6,410,062 to Callaghan, et al. describes a method of treating and preventing inflammatory disorders and related conditions by applying a topical composition comprising an effective amount of an extract of feverfew, where the extract is substantially free of α-unsaturated γ-lactone, and particularly substantially free of parthenolide.

U.S. Pat. No. 5,905,089 to Hwang, et al. describes the use of sesquiterpene lactones for treatment of severe inflammatory disorders; possible route of administration is transdermal. The term “transdermal” in this patent is described in a very specific manner that apparently does not contemplate topical application of a composition or a patch, stating that the composition may be “administered transdermally, for example in the form of a slow-release subcutaneous implant.” See column 6, line 26.

### SUMMARY OF THE INVENTION

It has been found that migrainous headaches and their associated symptoms may be treated by administration of a surprisingly small amount of parthenolide when said treatment is transdermally administered. The present invention has been found to be effective both as an acute (abortive) treatment and as a preventative (prophylactic) treatment. The combination of the correct dosage of active ingredient, together with the correct route of administration for these compositions results in a surprising degree of effectiveness for patients in need of acute and/or preventative treatment of migrainous headaches. Thus, surprisingly a material that has been administered to people for many years in various forms with widely varied effect on some maladies has been found to be as or more effective than prescription medication in the treatment of migrainous headaches when provided in compositions as described herein and administered using methods as described herein.

More particularly, the present invention provides a method of treating migrainous headaches and their associated symptoms, comprising transdermally administering a composition comprising parthenolide to a patient in need thereof in a total amount of from about 0.05 mg. to about 1.0 mg. of parthenolide in a two hour period. In another embodiment of the present invention, parthenolide is preferably transdermally administered over a 24 hour period, the total amount of parthenolide being about 0.05 mg. to about 1.0 mg. Various systems, including compositions and composites are disclosed herein for transdermal delivery of parthenolide to a patient. It is contemplated that such compositions and composites may contain an amount of parthenolide that exceeds the above indicated administration amounts. This is necessary because it is axiomatic that not all parthenolide present in any given matrix will be delivered to the patient. Thus, some parthenolide may be retained in the composition or composite at the time of removal from the patient. For purposes of the present invention, when an amount of parthenolide is indicated to be “administered,” it is contemplated that the transdermal delivery system is designed to delivery that amount to the patient during the stated time period, and is not intended as an indication of the total amount of parthenolide that is present in the matrix of the transdermal delivery system.

In a preferred embodiment of the present invention, migraine headaches and/or their associated symptoms are treated by administering a liquid composition comprising feverfew that contains parthenolide transdermally to a patient in need thereof in a total administered amount of from about 4 mg. to about 40 mg of feverfew extract in a two hour period. In another embodiment of the present invention, a plurality of doses of feverfew extract are administered transdermally over a 24 hour period, the total amount of feverfew extract not exceeding about 40 mg. Preferably, the individual doses of feverfew extract each does not exceed about 10 mg.

In one aspect of the present invention, preferably, each occurrence of a migrainous headache may be treated by administering parthenolide transdermally as described above. The treatment as described herein may be administered for the acute relief of a migrainous headache and its associated symptoms, from any time beginning with the first sign of impending migraine headache through such time as the migrainous headache is well underway. For certain individuals the first sign of an impending migrainous headache may be other than the first mild pain. Approximately 10% of those suffering migraine headaches experience classic migraine aura, most often changes in visual perception which are unique, and which the sufferer has learned to associate with impending migraine. Classic aura may also involve other senses, with or without visual sensations, such as a unique smell. Other individuals may realize that migraine onset is imminent by the occurrence of unusual aches, pains, or merely by subjective observation of their own mood or irritability.

In a particularly preferred aspect of the present invention, the treatment may also be used daily as a prophylactic treatment, or as needed as a prophylactic treatment. In a particularly preferred embodiment of the present invention, the individual user is administered a total administered amount of the composition as described herein in response to the occurrence of a predetermined event that that individual has identified as being associated with a higher incidence of migraine for them personally. Examples of such events include menstruation, climate changes, air travel, and the like. This prophylactic administration has been found to be surprisingly effective in preventing or minimizing the onset and occurrence of migrainous headaches and their associated symptoms. Surprisingly, the treatment may additionally be used to treat headaches associated with and resulting from the consumption of alcohol.

In one aspect of the present invention, convenient systems for administration of parthenolide, and as another embodiment, feverfew extract containing parthenolide, are provided wherein compositions are provided as a transdermal patch for transdermal administration. The transdermal patch preferably comprises parthenolide in an amount not exceeding about 1.0 mg. In another embodiment, the transdermal patch preferably comprises feverfew extract in an amount not exceeding about 40 mg, wherein the feverfew extract comprises parthenolide.

### BRIEF DESCRIPTION OF THE DRAWINGS

FIG. 1. shows a chart indicating time to onset of absorption for two delivery modes.

FIG. 2 shows a chart indicating time to peak plasma concentration for two delivery modes.

FIG. 3 shows a cross section of a simple adhesive patch having an impermeable backing layer, a parthenolide containing adhesive layer, and a release liner.

FIG. 4 shows a cross section of a matrix transdermal patch having an impermeable backing layer, a parthenolide source comprising a polymer matrix in which parthenolide is dispersed, a peripheral adhesive layer, a release liner. The patch may also contain a porous membrane.

FIG. 5 shows a cross section of a reservoir type transdermal patch having an impermeable backing, a parthenolide source, a porous membrane, an adhesive layer, and a release liner. The backing layer and the membrane are sealed at their peripheral edges, thus defining a parthenolide source.

### DETAILED DESCRIPTION

It has surprisingly been found that administration of a very small amount of parthenolide transdermally provides excellent treatment for migraine headaches and their associated symptoms, including acute relief of migraine headaches and their associated symptoms as well as prevention of the onset of migraine headaches and their associated symptoms. Because the active ingredients of the present composition are administered at a very low level, there is advantageously no need to obtain a prescription for use of this composition as an effective treatment of migraine headaches.

When an active agent is administered intravenously, intramuscularly, orally or mucosally, the initial level of the active agent in the blood rapidly rises to a maximum, which is generally much higher than the therapeutically effective level of the active agent. Sometimes initial levels of actives administered orally may reach toxic concentrations resulting in undesirable side-effects. This is known as “overdosing.” After the maximum level in the blood is reached, the concentration then falls slowly as the active is distributed, metabolized, excreted, or degraded. Eventually, the blood concentration of the active agent falls below the therapeutically effective level (i.e., there is “underdosing”). At this point, the active agent needs to be re-administered to achieve effectiveness. Maintaining the blood concentration of the active agent between the minimum therapeutically effective level and toxic levels is important. One way to achieve this is to administer lower active agent doses to the patient more frequently. This, however, is an unacceptable alternative in most instances, due to problems with patient compliance.

Transdermal delivery of parthenolide offers a means of circumventing the problems of overdosing and underdosing that may be associated with conventional parthenolide delivery methods. The transdermal delivery of parthenolide can be designed so that the rate of delivery of the parthenolide closely follows the rate of the clearance of the parthenolide from the environment, thus keeping constant levels of parthenolide in the blood, and reducing parthenolide waste and overdosing problems.

The term “pharmaceutically acceptable” or “therapeutically acceptable” refers to a substance which does not interfere with the effectiveness or the biological activity of the active ingredients and which is not toxic to the host or patient. The term “patient” refers to a mammal that is being treated. Preferably the patient is a human.

The ability to effectively treat migraine headaches and their associated symptoms using such low yet rapidly effective doses of active ingredient provides substantial benefits, including not only effective relief from migraine headaches and their associated symptoms, but also the drastic reduction in side effects which might otherwise be associated with said active ingredient. By far the most commonly reported side effect of feverfew use is the occurrence of mouth sores, primarily associated with chewing feverfew leaves, wherein it may be observed in up to 11% of such users. Mouth sores are occasionally accompanied by general inflammation of tissues in the mouth. Infrequently reported side effects of feverfew use include gastrointestinal side effects such as abdominal pain, indigestion, flatulence, diarrhea, nausea, and vomiting. Some users have reported that when daily use is stopped suddenly after long periods of use, rebound headaches may occur, along with anxiety, sleep disturbances, and muscle stiffness or pain (collectively referred to as “post-feverfew syndrome,” reported to occur in about 10% of migraine patients who abruptly stop taking feverfew after a long history of daily use). While most side effects are mild, it is nonetheless advantageous to avoid or reduce their occurrence to the greatest extent possible while still employing an effective dose. The methods and compositions of the present invention thus may provide an improved safety profile that may make the present invention most suitable to the average user, and particularly suitable for those with whom additional caution need be exercised, such as those who are sensitive to various medications or in the case of pediatric use, where additional cautions are generally warranted. The invention is particularly beneficial to those patients concerned about using large amounts of medication for treatment of ailments, and also to patients who wish to avoid the use of prescription medications, or who cannot afford the use of prescription medications. The low total administered amount of active ingredient and relatively small amount of total composition that is applied transdermally may additionally be of particular benefit to those treating acute migraine. This is because nausea and vomiting are frequently associated symptoms of migraine, making it often unpleasant and sometimes difficult or even impossible to administer medications in solid form or by way of larger volumes of liquids. The preparation is further beneficial to those wishing to employ migraine prophylaxis from time to time without the need to maintain daily intake of medication indefinitely.

As a new OTC medication, the present invention provides substantial benefits to migraineurs, not least of which may be the cost savings associated with a decreased reliance on expensive prescription medications and the reduction in the economic burden of migraine in the United States. There are also substantial advantages for those individuals who choose to rely on OTC medications for relief of migraine headache, namely the availability of another treatment option that may be more effective than currently available OTC products, or which may be associated with fewer side effects than currently available OTC products.

Some migraine headaches are preceded by aura or other symptom, whereby treatment of the symptom may prevent the further development of the migraine headache. The present invention provides surprisingly rapid relief, so that certain patients may avoid full onset of migrainous headaches and their associated symptoms through immediate administration of the present treatment upon onset of this symptom. In addition, many associated symptoms of migraine are found to be relieved, such as nausea, vomiting, photophobia and phonophobia (excessive/extreme sensitivity to light in one case and sound in the other). There is a growing body of knowledge which suggests that headache exists along a spectrum, with no rigid demarcation between “migraine” and “non-migraine,” and that many headaches previously thought to be “not migraine” are in fact “migrainous.” This may explain the fact that the present invention has been found to be effective in the treatment not only of headaches experienced by those not having been previously diagnosed with migraine, and whose headaches may not specifically qualify as migraine per the presently accepted diagnostic criteria of the International Headache Society, but also headaches not so diagnosed, including as but one example those associated with excessive alcohol use (e.g. “hangovers”).

Previously used treatments may be effective for some patients, but not for others, necessitating a lengthy and sometimes costly search by each person for the treatment that will be effective for that particular individual. Surprisingly, the present invention is effective for a large percentage of people who try this treatment. Thus, the present invention provides additional substantial benefit to the practitioner or individual who may discover conveniently and without delay those for whom this treatment is effective, searching amongst other alternatives in those cases where this medication proves insufficiently effective.

As noted above, preferably, migrainous headaches may be treated by administering a composition comprising parthenolide transdermally to a patient in need thereof in a total administered amount of about 0.05 mg to about 1.0 mg of parthenolide in a two hour period. More preferably, the total administered amount is from about 0.050 mg to about 0.7 mg of parthenolide, and most preferably, the total administered amount is from about 0.08 mg to about 0.6 mg of parthenolide in a two hour period.

Additionally, there is a growing desire to use naturally generated ingredients to provide treatments for disorders. Therefore, a particularly preferred embodiment of the present invention comprises administration of small quantities of feverfew extract. More specifically, a preferred method of treating migraine headaches comprises transdermally administering a composition comprising feverfew extract to a patient in need thereof in a total administered amount of from about 4 mg to about 40 mg of feverfew extract in a two hour period. More preferably, the total administered amount is from about 4 mg to about 20 mg of feverfew extract, and most preferably the total administered amount is from about 4 mg to about 12 mg of feverfew extract in a two hour period.

Feverfew extract is derived from the feverfew plant (Tanaecetum parthenium), which is also known, for example, as Chrysanthemum parthenium, Chrisanthemum parthenium, Pyrethrum parthenium, Tanacete parthenii herba or folium, Matricaria parthenoides, Matricaria parthenium, Leucanthemum parthenium, Matricaria parthenium, Spanish pellitory, Featherfew, Featherfoil, feather-fully, and by a number of common names, various of which are used throughout the world (Midsummer daisy, Bachelor's buttons, Altamisa, nosebleed, flirtwort, ague plant, devil daisy, feddygen fenyw (Welsh), maid's weed, Missouri snakeroot, mutterkaut (German), prairie-dock, vetter-voo, wild chamomile, grande camomille (French), Santa Maria (Spain), febrifuge plant.) The extract may be obtained by techniques known in the art using solvents such as petroleum spirits or polar organic solvents. See U.S. Pat. No. 5,384,121 to Rhodes, and also WO 94 06800; EP 0 553 658; WO 92 11857; GB 2,166,952; EP 98 041; WO 98 39018.

The extract of the feverfew plant contains parthenolide, and may additionally contain other components such as Polyynes, Flavonoids and Volatile oils including camphor, borneol and others, each of which may contribute to the therapeutic effect of the preparation disclosed herein. Feverfew also contains relatively large quantities of sesquiterpene lactones, primarily parthenolide.

In addition to parthenolide, feverfew is known to contain the following non-ubiquitous chemicals: 1-Beta-hydroxyarbusculin, 10-Epicanin, 8-Beta-reynosin, Apigenin-7-glucoside, Chrysanthemolide, Chrysanthemonin, Chrysartemin-A, Chrysartemin-B, Cosmosiin, L-Borneol, L-camphor, Mangoliolide, Reynosin, Santamarin, Tanaparthin, Tanaparthin-1-alpha, 4-alpha-epoxide, Tanaparthin-1-beta,4-beta-epoxide, tenetin 3-b-hydroxyparthenolide, seco-tanaparthenolide A, canin, artecanin, and balchanin.

Because feverfew extract may contain additional beneficial components, compositions comprising the extract of feverfew are generally preferred for use in the present invention as compared to compositions comprising a highly purified parthenolide that has been isolated from the additional components naturally occurring in feverfew extract. Preferred embodiments of the present invention use feverfew extract that has been standardized to contain a predetermined standardized parthenolide concentration of preferably not less than about 1.0%, and more preferably from about 1.2% to about 10%. Higher concentration parthenolide compositions may become readily available, which may advantageously reduce the amount of liquid required in the composition for delivery of the active to the user. While the source of parthenolide in compositions of the present invention is preferably feverfew as discussed above, it may alternatively be obtained from any number of other plant species, where it generally occurs in substantially lower concentrations. Such plant species include especially other members of the Compositae family, which include especially the many species of chrysanthemums, daisies, marigolds, chamomile, yarrow and aster. Parthenolide can also be obtained from tansy. Alternatively, parthenolide may be made by any appropriate synthetic route.

The composition to be used in the present invention may optionally comprise additional active ingredients. These active ingredients may also be provided as a treatment of migrainous headaches or may provide other physical benefits, provided that the treatment benefit of parthenolide and/or the feverfew extract is not adversely affected. In one aspect, preferably additional amounts of already present sesquiterpene lactones or additional sesquiterpene lactones are incorporated in the compositions of the present invention. Preferred such sesquiterpene lactones include especially those which are known to be contained in (naturally occur in) feverfew, such as 3-Beta-hydroxyparthenolide, seco-tanaparthenolide A, canin, artecanin, chrysanthemonin, chrysartemin A and B, santamarin and balchanin, as well as those occurring in other plant species such as encelin, leucanthin B, enhydrin, melampodin A, tenulin, confertiflorin, burrodin, psilostachyin A, costunolide, guaianolide, cinerenin, artemisinin, aristolactone, lactarorufin A, bilobalide, helenalin, furandiol. Sesquiterpene lactones in addition to parthenolide may be isolated from plants such as dandelion, burdock, butterburr, mugwort and sunflower plants, among others.

Compositions to be used in the present invention may optionally additionally comprise other naturally occurring components and extracts, including those as identified in the HPUS. Preferred additional components are extracts indicated for use in treatment of headaches, inflammation, nausea or anxiety. Particularly preferred additional components are the extracts of ginger and/or green tea, or the isolated components thereof. A particularly preferred isolated component of green tea is L-theanine.

Particularly preferred compositions of the present invention contain substantially no active ingredients other than those that are extractable from herbal sources. In a particularly preferred embodiment of the present invention, the compositions contain substantially no active ingredients other than those that are extractable from feverfew, ginger and green tea sources. In another particularly preferred embodiment, the compositions contain substantially no active ingredients other than those that are extractable from feverfew and ginger. Such compositions additionally may comprise non-pharmacologically active ingredients, such as thickeners, and carrier liquids. It has surprisingly been discovered that the use of only active ingredients that are extracted from herbs provide particular benefit to the user in being both effective in the treatment of migrainous headache, and also providing natural healing conditions particularly suited to the well being of patients. Surprisingly, these natural ingredients have been found to be effective in the indicated dosage ranges when administered in a transdermal regimen as described herein. Such compositions contain parthenolide in the amounts as discussed earlier, and preferably contain less than about 400 mg of any given natural active ingredient per dose.

In particular, the combination of feverfew extract together with ginger extract is particularly effective in providing relief from migrainous headaches when provided and administered as taught herein. It has been observed that the combination of these extracts in the present compositions in particular provide synergistic effect in relieving both the pain and the nausea and/or general discomfort associated with migrainous headaches that is beyond a mere additive effect of these extracts. Incorporation of additional herbal extracts in amount as indicated herein, when delivering the composition as a transdermal administration, also provide excellent total beneficial effects of the composition exceeding what would be expected from the herbal and homeopathic literature.

In a particularly preferred embodiment, the composition to be used in the present invention additionally comprises ginger extract at a total administered amount preferably not exceeding about 400 mg, and more preferably not exceeding about 250 mg of ginger extract in a two hour period. Particularly preferred compositions comprise ginger extract as about 0.1-10% of the total composition.

In another particularly preferred embodiment, the composition additionally comprises L-theanine, either as an isolated component or as a constituent of green tea extract, but in either case at a total administered amount preferably not exceeding about 400 mg of L-theanine in a two hour period. Particularly preferred compositions comprise L-theanine as about 0.1-10% of the total composition.

In a particularly preferred embodiment of the present invention, the compositions of the present invention are provided at a pH of from about 2.0 to about 6.5, more preferably at a pH of from about 2.5 to about 6.0, and more preferably at a pH of from about 3 to about 5. Surprisingly, it has been found that compositions having a pH of the range indicated above are much more effective than compositions outside of the indicated range. Various pH adjusters may be used to adjust the pH of the composition to the desired level. Examples of suitable pH adjusters include hydrochloric acid, citric acid, phosphoric acid, acetic acid, tartaric acid, sodium hydroxide, potassium hydroxide, sodium carbonate, sodium hydrogen carbonate, boric acid, sodium borate, and the like. Preferably, the pH of the composition is adjusted to be acidic using ascorbic acid.

Preferably, the composition is buffered by a pharmaceutically acceptable buffer. Examples of buffering agents include borate buffers, citrate buffers, phosphate buffers, tartarate buffers, acetate buffers, carbonate buffers, and amino acid salts, etc. Most preferably, the buffer is sodium citrate.

Preferably, the liquid composition comprises 0.05% -10% feverfew extract, 0.1-10% ginger extract, 0.1-10% L-theanine, and 10-98% water. Preferably, the feverfew extract has a standardized parthenolide concentration of not less than about 1.0%, and more preferably from about 1.2% to about 10%.

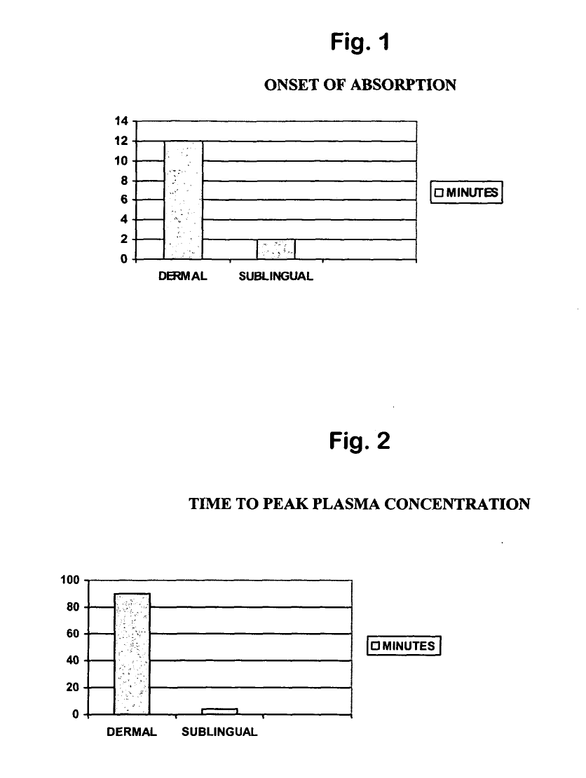

In situations where very rapid onset of action is desired along with a sustained blood level, an initial dose may be provided by means of sublingual administration of parthenolide, which provides an immediate and rapid rise in blood levels of the desired composition. Transdermal delivery as presently described may then be employed in combination with the rapid sublingual delivery mechanism, thereby maintaining blood levels of the active agents in the therapeutic range. As shown in FIG. 1, the amount of time required for onset of absorption of a representative active (nitro glycerin) when an active ingredient is delivered via a sublingual administration route is very short (about 2 minutes), while the time required for onset of absorption when the active is delivered via transdermal delivery is much longer (about 11 minutes). As shown in FIG. 2, the amount of time required to achieve peak plasma concentration when a representative active ingredient (nitro glycerin) is delivered via a sublingual administration route is also very short (about 5 minutes), while the time required to achieve peak plasma concentration when the active is delivered via transdermal delivery is much longer (about 90 minutes). Thus, a particularly preferred embodiment of the present invention sublingual and transdermal administration as taught herein, so that one may achieve rapid active ingredient levels that are then sustained over many hours.

In a particularly preferred aspect of the present invention, an aqueous composition comprising parthenolide is administered sublingually to a patient in need thereof in a total sublingually administered amount of from about 0.05 mg to about 1.0 mg of parthenolide in a two hour period, and additionally parthenolide is transdermally administered to the patient in a total transdermally administered amount of from about 0.05 mg to about 1.0 mg of parthenolide in the same two hour period. Most preferably, the sublingual administration as described above is administered, and a transdermal system is applied to the skin of the patient within about 10 minutes prior to or after the sublingual administration for longer term administration of parthenolide.

The above described combination administration technique is particularly preferred when used in response to the first sign of impending migraine headache or in response to or anticipation of the occurrence of a predetermined event that that individual has identified as being associated with a higher incidence of migraine for them personally. Examples of these events are discussed above. Of particular significance is the ability to apply effective treatment of migrainous headaches around the time of menses.

Particularly preferred compositions and methods of sublingual delivery of parthenolide are disclosed in [attorney docket No. GSC0002/US/2], Mitchell et. al. filed on even date with the present application, the disclosure of which is incorporated herein by reference. Most preferably, the sublingual compositions comprise parthenolide in an amount of from about 0.01 mg/ml to about 0.25 mg/ml.

Most preferably, the composition is administered as a first sublingual application of a first composition comprising not exceeding about 0.5 mg of parthenolide, or alternatively, not exceeding about 20 mg of feverfew, which first composition is held in place under the tongue for a predetermined time, preferably about 30 seconds, or more preferably about 60 seconds or more, after which the composition is swallowed. Most preferably, the composition is circulated or “swished” around the mouth by the patient prior to swallowing. Surprisingly, this apparently minor addition to the procedure noticeably increases the effect of the composition in the treatment. A second composition not exceeding about 0.5 mg of parthenolide, or alternatively, not exceeding about 20 mg of feverfew, is then applied and held under the tongue for a predetermined time, preferably about 30 seconds, or more preferably about 60 seconds or more, after which the second composition also is swallowed. Again, preferably the composition is circulated or “swished” around the mouth by the patient prior to swallowing. Alternatively, the total administered amount not exceeding about 1.0 mg of parthenolide, or alternatively about 40 mg of feverfew extract, may be divided among three or more compositions for sequential application as described above.

In a particularly preferred aspect of the present invention, the transdermal delivery system may be designed to delivery parthenolide at the indicated rate for an extended period. Thus, preferably the transdermal delivery system will administer parthenolide at the indicated rates for periods of 24 hours, or from about 1 to about 3 days, about 3 to about 7 days for from about 1 week to about 4 weeks. Longer rates of delivery of parthenolide are also contemplated. The extended controlled dose delivery of parthenolide as described herein provide particular benefit to the user in a prophylactic uses, and additionally in prevention of headache rebound. Transdermal delivery systems capable of delivering an effective dose of parthenolide over a period of about 3-7 days are particularly advantageous for combating migrainous headaches associated with menses.

In addition to the advantage of being able to control the delivery rate of parthenolide, transdermal delivery also provides a comfortable, convenient and non-invasive method of administering parthenolide. Gastrointestinal irritation and other side-effects associated with oral parthenolide delivery may be reduced or eliminated, and patient anxiety regarding invasive delivery methods, such as needles, is also eliminated.

Further, the transdermal administration avoids the “first pass effect,” which often results when a medication is administered orally and thus has to pass through various organs, including the liver, before reaching the affected area of the body. These organs can absorb or chemically alter significant quantities of the passing medication, thus requiring that large excess quantities of the medication by administered initially to insure that an effective quantity of the medication will ultimately reach the affected area of the body.

In one embodiment of the present invention, compositions are provided in the form of a lotion cream or other spreadable or moldable material. Using these formulations, parthenolide may be effectively topically administered by application of the cream to many bodily areas where a patch either will not fit or cannot be shaped to conform to the skin contours. Such compositions may be readily prepared by the routineer in the field, by consulting with established formularies and substituting the indicated active ingredients as taught herein.

For example, an appropriate cream composition may be formulated by forming an organogel from lecithin and isopropyl palmitate, as disclosed in U.S. Pat. No. 5,837,289. Suitable gel structures thus may be formed and used as the base for a cream composition. Parthenolide is preferably solubilized with a solvent, such as water, alcohol or other appropriate solvent, and mixed into the matrix to formulate an appropriate composition for application to the dermis of a patient, for transdermal delivery of parthenolide to the patient.

Additional components, such as cosmetic agents, binders, thickeners, preservatives and fragrances may be present in the topically applied cream or other moldable or spreadable material.

Cosmetic agents which may be used in the compositions of this invention may include, but are not limited to those agents which prevent potential skin irritation, such as emollients, vitamins and antioxidants (e.g., vitamin E) and herbal extracts (e.g., aloe vera). Further, the cosmetic agents may include humectants, antioxidants/preservatives, plant extracts, surface active agents, and the like. Examples of humectants include glycerol, sorbitol, propylene glycol, ethylene glycol, 1,3-butylene glycol, polypropylene glycol, xylitol, maltitol, lactitol, oat protein, allantoin, acetamine MEA, hyaluronic acid and the like. They may be used either singly or in combination.

Binders or thickeners may be used in the compositions of this invention to provide substantivity and physical stability to the compositions. Binders or thickeners suitable for use in the compositions of this invention include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickeners such as natural gums and synthetic polymers, as well as coloring agents and fragrances also are commonly included in such compositions.

Examples of preservatives which may be used in the compositions of this invention include, but are not limited to, salicylic acid, chlorhexidine hydrochloride, phenoxyethanol, sodium benzoate, methyl para-hydroxybenzoate, ethyl para-hydroxybenzoate, propyl para-hydroxybenzoate, butyl parahydroxybenzoate and the like.

Examples of fragrances which may be used in the compositions of this invention include menthol, anethole, carvone, eugenol, limonene, ocimene, n-decylalcohol, citronellol, a-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

The compositions of the present invention may be prepared in a number of forms for topical application to a patient. For example, the composition may be applied in a gel, cream, ointment, shampoo, scalp conditioners, liquid, spray liquid, paint-/brush-on preparation or aerosol. In addition the composition may be impregnated on a bandages, hydrocolloid dressing, treatment patch or on cloth wipe products, such as baby wipes or facial wipes.

The compositions of this invention may be in the form of emulsions, such as creams, lotions and the like. Such compositions may have more than one phase and may include surface active agents which enable multiphase emulsions to be manufactured.

Optionally, a cover sheet may be applied over the applied cream or spreadable material containing parthenolide, and further the cover sheet may be secured with an adhesive sheet or strip to protect against undesired exposure of the applied cream to the elements.

Preferably, transdermal application of Active is carried out by use of a transdermal composite, commonly referred to as a “patch.” In this embodiment, the transdermal composite is a preconstructed composite capable of adhering to the dermis of a patient, having an effective amount of Active that can be delivered from the transdermal composite, and a protective overlay material that substantially prevents undesired loss of Active to the air or to surfaces that may contact the outer portion of the transdermal composite.

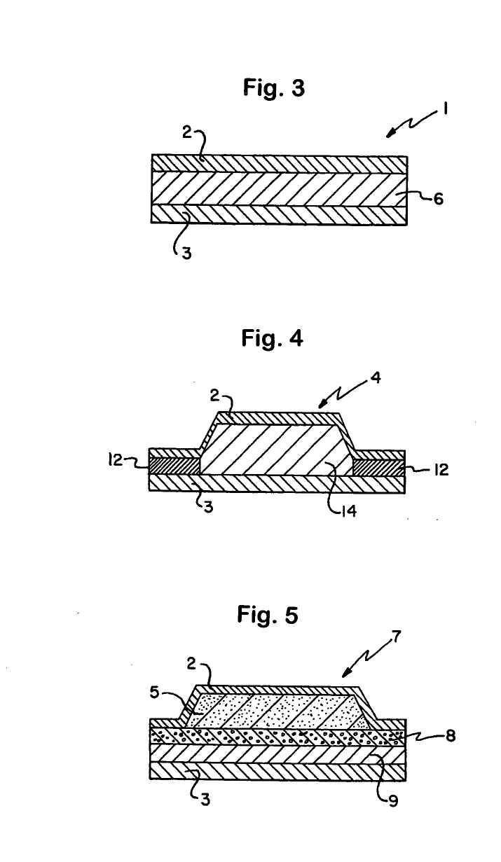

The transdermal composite may be provided in a number of configurations, as exemplified in FIGS. 3 through 5.

In one embodiment of the invention, the transdermal patch for the delivery of parthenolide (referred to as the Active) is a simple adhesive patch, as shown in FIG. 3. In FIG. 3, the patch 1 comprises an impermeable backing layer 2, a release liner 3, and an Active/adhesive containing matrix 6. The impermeable backing layer 2 defines the top of the delivery device, i.e., the side furthest away from the skin when the device is in use. The backing forms an occlusive layer that prevents the loss of Active and/or enhancers to the environment and protects the patch from contamination from the environment. The backing layer may be opaque so as to protect the Active from light.

The backing layer can be made from standard commercially available films for medical use, such as those supplied by 3M Corporation, St. Paul, Minn.; Dow Chemical, Midland, Mich.; or AF Packaging, Winston-Salem, N.C. Suitable materials which can be used to form the backing layer include films or sheets of polyolefin, polyester, polyurethane, polyvinyl alcohol, polyvinylidene, polyamide, ethylene-vinylacetate copolymer, ethylene-ethylacrylate copolymer, and the like, metal-vapor deposited films or sheets thereof, rubber sheets or films, expanded synthetic resin sheets or films, unwoven fabrics, fabrics, knitted fabrics, paper, and foils. These materials can be used individually or as laminates. These films can be pigmented or metalized.

In some aspects of the invention, the patch may include a peel strip or release liner 3 to cover the surface of the pressure-sensitive adhesive during storage, and prevent evaporative loss of the Active or enhancer(s). The release liner may be formed with dimples for decreasing contacting surface with the adhesive layer, and it may also be formed with a pull-tab for making it easier for removing it from the device.

The peel strip may be made from any impermeable film, such as is specified for the backing layer. Additionally it may be made from metal foil, Mylar™ film, polyethylene terephthalate, or any material normally used for this purpose in the art that is compatible with the Active and the chosen adhesive. Examples of suitable compositions for the release liner include siliconized polyester, poly (1,1-dihydroperfluoroctylmethacrylate), fumed silica in silicone rubber, end-capped siliconized polyethylene terephthalate, polytetrafluoroethylene, cellophane, a film of polyvinyl chloride having titanium dioxide dispersed therein, and the like.

In the simple adhesive matrix patch of FIG. 3, the Active source layer is comprised of the Active and an adhesive, the layer attaching directly to the skin of the patient after the peel strip or release liner is removed. In the preferred embodiments of the invention, the Active source layer also comprises one or more enhancers.

Generally, the selection of the adhesive is important to the proper functioning of the transdermal delivery device. This is particularly true if a plasticizer-type enhancer is placed in the adhesive layer. Specifically, the adhesive layer must retain its functioning properties in the presence of the plasticizer-type and solvent-type enhancers, as well as upon exposure to the Active.

Alternatively, the adhesive may comprise matrix regions interspersed throughout the adhesive, wherein the matrix regions comprise Active. These matrix regions act as small reservoirs from which Active is released.

FIG. 4 shows a matrix patch, an alternative embodiment of the transdermal patch of the invention. In FIG. 4, the patch 4 comprises an impermeable backing layer 2, a release liner 3, a matrix layer 14 comprising a matrix in which Active is dispersed, and a peripheral adhesive layer 12. The matrix may be a polymer matrix, or a gel or cream in which the Active resides. In some embodiments, the patch may also include an optional porous membrane layer. In yet other embodiments, the patch may have an adhesive layer that is co-extensive with the skin facing surface of the patch. The matrix layer 14 comprises the Active, and one or more enhancers dispersed in a polymeric matrix.

The matrix layer may also comprise additional components such as diluents, stabilizers, vehicles, biocides, antioxidants, anti-irritants and the like. A preferred embodiment of the matrix patch is a matrix patch with a peripheral adhesive annular ring and an Active source having a hydrogel matrix or a foam matrix.

A further embodiment of the invention is the reservoir type patch which allows a higher loading level of active material, and usually, a higher loading level of enhancer. An example of the reservoir type patch is shown in FIG. 5. The patch 7 is comprised of an impermeable backing layer 2 which is sealed at its periphery to an inert membrane 8, thereby defining between these two layers an Active source 5. An adhesive layer 9 is affixed to the skin facing side of the patch. The patch also comprises a release liner 3. The Active source contains the Active, and optionally one or more enhancers or gelling components. Thus, in the reservoir type patches, a membrane separates the Active reservoir from the adhesive layer. In some embodiments the membrane is a non-rate controlling membrane. According to the present invention, a non-rate controlling membrane is one in which the rate of permeation of the enhancer(s) and Active through the membrane is greater than their permeation rate through the skin or any other portion of the device (typically two to five times greater or more). Thus, a non-rate controlling membrane is extremely permeable to the enhancer(s) and the Active contained in the reservoir.

In other embodiments, the membrane may be a rate-controlling membrane. As used herein, a rate-controlling membrane is one in which the rate of permeation of the enhancer(s) and the Active through the membrane is less than or equal to their permeation rate through the skin or any other portion of the device. Rate-controlling membranes are described, for example, in U.S. Pat. Nos. 4,460,372 and 4,379,454. The membrane may comprise a microporous or porous material. Microporous membranes have a distinct pore structure with pores ranging in diameter from approximately 0.08 to 0.5 microns, preferably from about 0.1 and 0.4 microns, and more preferably from about 0.2 and 0.4 microns. Examples of suitable microporous membranes include polyethylene and polypropylene films, nylon, and nitrocellulose film.

In the reservoir type patches, the membrane and the backing layer are sealed at their peripheral edges to form the Active reservoir. This seal should be substantially fluid-tight to prevent Active leakage from the reservoir through the seal between the backing layer and the membrane. As used herein, the term “peripheral edges” of the membrane and backing layers refer to the areas that are sealed together to define the Active reservoir. Therefore, extraneous membrane and backing layer material may extend outwardly from the Active reservoir and peripheral edge.

The Active reservoir contains a solution, suspension, or gel of the Active and the permeation enhancers, as well as diluents, such as water, and vehicles or other additives. The Active can be dispersed in the solution, suspension, or gel in either a dissolved or undissolved state.

A gelling agent may be incorporated into the reservoir or matrix to increase the viscosity and rheological characteristics of the Active and enhancers. The gelling agent comprises a pharmaceutically-acceptable material that is capable of increasing viscosity of the reservoir solution. Typically, the Active delivery devices described herein will employ cellulosic materials as the gelling agent. Examples of suitable cellulosic materials include cellulose, cellulose derivatives, alkylcellulose, hydroxy-(lower alkyl) cellulose derivatives where the alkyl group contains one to six carbons, carboxyalkylcellulose and the like. Other gelling agents include PVP, CMC, Klucel, alginates, kaolinate, bentonite, or montmorillonite, other clay fillers, stearates, silicon dioxide particles, carboxy polymethylene, ethylene maleic anhydride, polyacrylamide, and poly (methyl vinyl ether maleic anhydride.)

The reservoir or matrix layer also may include diluents, stabilizers, vehicles, biocides, antioxidants, anti-irritants and the like. For example, water is frequently utilized as a diluent in the reservoir type patches. Typically water will be present in the reservoir in an amount not greater than about 50 wt %, based on the reservoir fill solution; preferably, not greater than 40 wt %. Other diluents which will frequently find use in the Active delivery devices described herein include glycerine and propylene glycol.

A pressure-sensitive adhesive layer is affixed to the membrane opposite to the backing layer. In one embodiment, the adhesive layer should interact minimally with the Active. In another embodiment, the adhesive may comprise Active for additional delivery of Active to the user. The adhesive should adhere firmly to the membrane, but removably to the release liner. The device should stick securely to the wearer for extended periods, yet be removed at the desired time with minimum discomfort. The device should not give rise to undue skin irritation, allergic reactions or other dermatological problems. These properties must be maintained from the time of patch manufacture, throughout storage, and up to and throughout the time of application.

An alternative embodiment of the reservoir patch has a peripheral adhesive, wherein the area of the adhesive layer is not co-extensive with the active releasing area of the patch, but rather forms an annular ring around the active releasing area of the patch. The delivery of the Active thus is not primarily through the adhesive layer of the patch, although some lateral diffusion may occur within the patch, resulting in delivery of active substance through the adhesive at the periphery of the patch. The shape of the peripheral adhesive region will vary with the shape of the patch, but will generally comprise the outer perimeter of the patch, in order that an adequate adhesive seal is maintained between the skin and the patch to prevent the patch from falling off. The percentage of the patch that comprises the peripheral adhesive portion depends on the type of adhesive, the type of backing layer, the length of time the patch will be worn, and the weight and loading of Active in the patch. Such determinations will be apparent to the skilled artisan.

Prior to use, the patches typically are stored in laminate foil pouches, both to prevent contamination and to avoid Active and/or enhancer(s) loss. Such pouches are standard in the industry, and therefore may be selected by the routineer in this art. The patch may be assembled by any of the techniques known in the art for producing transdermal patches. The patches may be of various shapes, but the round shape is preferred as it contains no corners and thus is less easily detached from the skin.

For adequate skin penetration of most Actives, a chemical permeation enhancer may be desired. As used herein, the term “enhancer” is meant to encompass any enhancer or combination of enhancers that increases the flux of a substance across a mammalian stratum corneum. There are numerous possible permeation enhancers that can be used and they are typically categorized into two groups, solvent-type enhancers and plasticizing-type enhancers.

Plasticizer-type enhancers refers to fatty acids, fatty acid esters, fatty alcohols and similar hydrophobic compounds that are capable of increasing the permeability of Actives to the stratum corneum. Without limiting the scope of the present invention, the following is proposed as the mechanism of action of the plasticizer-type enhancers. It is believed that the function of the plasticizer-type enhancers is to migrate into the upper stratum corneum layers of the skin and disrupt the lipids which occupy the extracellular spaces of the stratum corneum. The stratum corneum layer, although only 25-50 microns thick, is the principal barrier to transdermal permeation. The plasticizer-type enhancers that migrate into the skin serve to increase the mobility and diffusion of the Active into the skin.

Plasticizer-type enhancers generally will have a molecular weight of greater than 150 but less than 1000. In addition, the plasticizer-type enhancers should also be relatively water insoluble or they will leach into the subcutaneous tissue layers below the stratum corneum. Thus, plasticizer-type enhancers with water solubility of less than 0.5 wt % are preferred, and more preferably 0.2 wt % or less.

A preferred group of plasticizer-type enhancers includes lower alkyl and alkoxy esters of pharmaceutically acceptable fatty acids, fatty acid esters, fatty alcohols, and similar hydrophobic compounds. As used herein, the term ‘lower alkyl and lower alkoxy’ refers to alkyl and alkoxy groups having up to and including 7 carbon atoms and preferably, up to and including 4 carbon atoms. Some examples of alkyl groups include methyl, ethyl, propyl, isopropyl, n-butyl, tertiary butyl, pentyl, isopentyl, neopentyl, hexyl, isohexyl and heptyl. Some examples of alkoxy groups include the oxides corresponding to the above alkyl groups. Examples of suitable fatty acid esters include saturated or unsaturated fatty acid esters, including isopropyl myristate, isopropyl palmitate, and the methyl and ethyl esters of oleic and lauric acid. Suitable fatty alcohols include stearyl alcohol and oleyl alcohol. Examples of suitable fatty acids include saturated and unsaturated fatty acids, including oleic acid, lauric acid, myristic acid, palmitic acid, stearic acid, linoleic acid, and palmitoleic acid. In addition, many other compounds can also serve as plasticizer-type enhancers, such as diethyl hexyl phthalate, octyldocecyl myristate, isostearyl isostearate, caprylic/capric triglycerides including polyethylene glycol esters of caprylic/capric acids, propylene glycol laurate (Lauroglycol), Miglyol (propylene glycol diester caproic, caprylic, capric, lauric acid), Lexol PG-865 (propylene glycol diester decanoic, octanoic acid), propylene glycol myristate (mirpyl), corn oil polyethylene glycol-6 esters (Labrafil M2124CS), polyethylene glycol-8 caprylic capric glycerides (Labrasol), caprylic/capric triglycerides (Labrafac Lipophile WL 1349), caprylic/capric triglyceride polyethylene glycol-4 esters (Labrafac Hydro WL1219 available from Gattefosse, Westwood N.J.), glyceryl oleate, hexamethyldisiloxane, m dimethicone, cyclomethicone, squalene, mineral oil, macrocyclic ketones/lactones, plant extracts such as Chrodarom Calendula O or Chrodarom Chamomile O, and various oils including wintergreen, jojoba oil, or eucalyptol (cineole). A preferred plasticizer-type enhancer for use with the mesylate salt of Rec 15/2739 include caprylic/capric acids triglyceride PEG-4 esters, available as Labrafac Hydro WL 1219, (Gattefosse, Westwood, N.J.) which contains a mixture of saturated polyglycolyzed glycerides consisting of glycerides and polyethylene glycol esters of caprylic and capric acids. One of skill in the art would appreciate that the plasticizer-type enhancers may be used alone or in combination. A particularly preferred enhancer combination including the caprylic/capric triglycerides for use with the mesylate salt of Rec 15/2739 is an enhancer vehicle consisting essentially of ethanol:caprylic/capric triglycerides polyethylene glycol-4 ester:propylene glycol:isopropyl myristate in a 1:1:1:1 ratio.

As used herein, “solvent-type enhancer” generally refers to relatively hydrophilic compounds having molecular weights of less than about 200 that are capable of increasing the permeability of Actives to the stratum corneum. Solvent-type enhancers typically exhibit solubility parameters between about 10 and 24, and preferably between about 10 and 18. Solvent-type enhancers are often better enhancers because they generally provide higher flux rates for a given permeant than plasticizer-type enhancers. Typically, the solvent type enhancers will comprise a pharmaceutically-acceptable lower alkyl alcohol, aryl alcohol, or polyol, for example, ethanol, propanol, butanol, benzyl alcohol, glycerin, or propylene glycol. In some embodiments, the solvent-type enhancer is a 2-pyrrolidone or alkyl derivative thereof, such as N-methyl-2-pyrrolidone, 3-hydroxy-N-methyl-2-pyrrolidone, and pyroglutamic acid esters.

Other embodiments may employ an alkyl ether, such as ethylene, polyethylene or propylene glycol ether, as the solvent type enhancer. Preferred examples of ethylene glycol ethers include, but are not limited to, ethylene glycol monoalkyl ethers, such as ethylene glycol monomethyl ether (also known as methyl cellosolve), ethylene glycol dialkyl ethers, such as ethylene glycol dimethyl ether (also known as dimethyl cellosolve), and ethylene glycol monoalkyl ether esters, such as ethylene glycol monoethyl ether acetate (also known as cellosolve acetate). Preferred examples of polyethylene glycol ethers include, but are not limited to, diethylene glycol monoalkyl ethers, such as diethylene glycol monobutyl ether (also known as butyl ethyl Cellosolve or butyl carbitol), diethylene glycol dialkyl ethers; and diethylene glycol monoalkyl ether esters, such as diethylene glycol monoethyl ether acetate (also known as Carbitol acetate), and transcutol (diethylene glycol monoethyl ether).

Preferred solvent type enhancers have a molecular weight of less than about 150. They are also relatively hydrophilic, generally being greater than 2 wt % soluble in water, and are preferably greater than 10 wt % soluble in water. Most preferred solvent type enhancers are completely water miscible. One of skill in the art would appreciate that the solvent type enhancers may be used alone or in combination.

The percentage by weight of the Active in the solution, hydrogel or matrix may be varied according to the desired loading of the finished patch.

All patents, patent documents, and publications cited herein are incorporated by reference as if individually incorporated. Unless otherwise indicated, all parts and percentages are by weight. The foregoing detailed description has been given for clarity of understanding only. It will be appreciated that numerous modifications and variations of the invention are possible in light of the above teachings, and therefore the invention may be practiced otherwise than as particularly described.

1. A method of treating migrainous headaches, comprising administering a composition comprising parthenolide transdermally to a patient in need thereof in a total administered amount of from about 0.05 mg to about 1.0 mg of parthenolide in a two hour period.

2. The method of claim 1, wherein said total administered amount is from about 0.05 mg to about 0.7 mg of parthenolide in a two hour period.

3. A method of treating migrainous headaches, comprising administering a composition comprising parthenolide transdermally to a patient in need thereof in a total administered amount of from about 0.05 mg to about 1.0 mg of parthenolide in a 24 hour period.

4. The method of claim 1, wherein said composition additionally comprises ginger extract.

5. The method of claim 4, wherein said ginger extract is administered at a total administered amount not exceeding about 250 mg of ginger extract in a two hour period.

6. The method of claim 1, wherein said composition additionally comprises green tea extract.

7. The method of claim 1, wherein said composition additionally comprises L-theanine.

8. The method of claim 7, wherein said L-theanine is administered at a total administered amount not exceeding about 400 mg in a two hour period

9. The method of claim 1, wherein said composition contains substantially no active ingredients other than those that are extractable from herbal sources.

10. The method of claim 1, wherein said composition contains substantially no active ingredients other than those that are extractable from feverfew, ginger and green tea sources.

11. The method of claim 10, wherein said composition contains less than about 400 mg of any given natural active ingredient per dose.

12. A method of treating hangovers, comprising administering a composition comprising parthenolide transdermally to a patient in need thereof in a total administered amount of from about 0.05 mg to about 1.0 mg of parthenolide in a two hour period.

13. A method of treating migrainous headaches, comprising
a) administering a composition comprising parthenolide sublingually to a patient in need thereof in a total sublingually administered amount of from about 0.05 mg to about 1.0 mg of parthenolide in a two hour period.
b) administering parthenolide transdermally to the patient in need thereof in a total transdermally administered amount of from about 0.05 mg to about 1.0 mg of parthenolide within the same two hour period.

14. A method of treating migrainous headaches, comprising
a) identifying the first sign of an impending migrainous headache in a patient;
b) responsive to the occurrence of the first sign in the patient, administering a composition comprising parthenolide sublingually to the patient in a total sublingually administered amount of from about 0.05 mg to about 1.0 mg of parthenolide in a two hour period; and
c) administering parthenolide transdermally to the patient in a total transdermally administered amount of from about 0.05 mg to about 1.0 mg of parthenolide within the same two hour period.

15. A method of treating migrainous headaches, comprising
a) identifying a predetermined event that a patient associates with a higher incidence of migraine for them personally,
b) in response to or anticipation of the occurrence of the event, transdermally administering a composition comprising parthenolide transdermally to the patient in a total transdermally administered amount of from about 0.05 mg to about 1.0 mg of parthenolide within a two hour period.

16. The method of claim 15, wherein parthenolide is administered transdermally at a rate of about 0.05 mg to about 1.0 mg of parthenolide every two hours for the duration of the predetermined event.

17. The method of claim 16, wherein the predetermined event is menses.

18. A topically applied composition for transdermally delivering parthenolide to a patient in need thereof, said composition comprising parthenolide in an amount sufficient to administer to the patient a total transdermally administered amount of from about 0.05 mg to about 1.0 mg of parthenolide within a two hour period.

19. The composition of claim 18, said composition being a lotion.

20. A transdermal delivery patch comprising parthenolide, said patch being engineered to deliver parthenolide to a patient transdermally at a rate of about 0.05 mg to about 1.0 mg of parthenolide in a two hour period.

21. The transdermal delivery patch of claim 20, said patch comprising a backing layer and an adhesive matrix layer comprising parthenolide.

22. The transdermal delivery patch of claim 20, said patch comprising a backing layer, a matrix layer comprising parthenolide on one surface of said backing layer, and a peripheral adhesive layer positioned to adhere the patch to a patient while allowing contact of at least a portion of the matrix layer to the patient.

23. The transdermal delivery patch of claim 20, said patch comprising a backing layer, a reservoir comprising parthenolide on said backing layer, an inert membrane on the opposite side of the reservoir from the backing layer, and an adhesive layer positioned to adhere the patch to a patient.

24. The patch of claim 23, wherein the inert membrane is a rate controlling membrane for regulating the rate of transmission of parthenolide from the patch to be transdermally administered to the patient.

25. The patch of claim 23, wherein the adhesive layer completely covers one surface of the inert membrane, so that parthenolide migrates through the adhesive layer to be transdermally administered to the patient.

26. A transdermal delivery patch comprising parthenolide, said patch being engineered to deliver parthenolide to a patient transdermally at a rate about 0.05 mg to about 1.0 mg of parthenolide in a 24 hour period.
